(19) United States
(12) Patent Application Publication
(10) Pub. No.: US 2024/0363203 A1
(43) Pub. Date: Oct. 31, 2024

(54) METHODS AND SYSTEMS FOR FUEL DESIGN USING MACHINE LEARNING

(71) Applicant: SAUDI ARABIAN OIL COMPANY, Dhahran (SA)

(72) Inventors: Balaji Mohan, Dhahran (SA); Junseok Chang, Dhahran (SA)

(73) Assignee: SAUDI ARABIAN OIL COMPANY, Dhahran (SA)

(21) Appl. No.: 18/306,610

(22) Filed: Apr. 25, 2023

(51) Int. Cl. G16C 20/70; G16C 20/30

(52) U.S. Cl. CPC G16C 20/70; G16C 20/30

(57) ABSTRACT

A system that includes a molecular descriptor generator configured to receive a simplified molecular-input line-entry system (SMILES) representation of a molecule and output a molecular descriptor for the molecule. The system further includes a trained chemical super learner model configured to receive the molecular descriptor and output a property prediction for the molecule, wherein the trained chemical super learner model is composed of a weighted average of one or more trained machine-learned models. The system further includes a computer that includes one or more computer processors configured to receive a first production example with a SMILES representation of a first molecule, process the first production example with the molecular descriptor generator to produce a first molecular descriptor, and process, with the trained chemical super learner model, the first molecular descriptor to determine a first prediction for a property of the first molecule.

### BACKGROUND

The creation of novel fuels with improved properties, such as increased performance (e.g., energy potential), stability over a broad range of environmental conditions, and reduction of noxious products, is a complex, costly, and difficult process. This is, in part, due to the fact that changes in the molecular structure of a fuel compound can have drastic effects on the characteristics and behavior of the fuel compound. To date, there is no physics-based predictive method, or first principles model, that can relate the molecular structure of a fuel compound to a property of the fuel compound. As a result, discovery of enhanced fuels generally encompasses proposing a fuel compound, synthesizing the proposed fuel compound (if possible), and measuring properties of the proposed fuel compound with a series of characterization tests. In practice, the process of synthesizing and testing a proposed fuel compound is extremely costly as, among other things, synthesis paths may need to be determined and characterization tests are usually performed at slowly increasing scales for safety considerations. The issues and complexities of novel fuel design are further compounded when considering the number of potential fuel compounds. Even restricting the chemical space of potential fuels to only include permutations of carbon, hydrogen, nitrogen, and oxygen (CHNO) atoms, the number of potential compounds is arguably near infinite. Consequently, it is not feasible to synthesize and test all potential fuel compounds to discover a fuel compound with desired properties that are markedly improved over the state-of-the-art. Accordingly, there exists a need to predict fuel properties given a proposed molecular structure for high-throughput screening of potential fuel compounds.

### SUMMARY

This summary is provided to introduce a selection of concepts that are further described below in the detailed description. This summary is not intended to identify key or essential features of the claimed subject matter, nor is it intended to be used as an aid in limiting the scope of the claimed subject matter.

Embodiments disclosed herein generally relate to a system that includes a molecular descriptor generator configured to receive a simplified molecular-input line-entry system (SMILES) representation of a molecule and output a molecular descriptor for the molecule. The system further includes a pre-processor configured to receive the molecular descriptor from the molecular descriptor generator and output a pre-processed molecular descriptor, wherein the pre-processor comprises a set of previously determined pre-processor parameters. The system further includes a trained chemical super learner model configured to receive the pre-processed molecular descriptor from the pre-processor and output a property prediction for the molecule, wherein the trained chemical super learner model is composed of a weighted average of one or more trained machine-learned models. The system further includes a computer that includes one or more computer processors configured to receive a first production example comprising a SMILES representation of a first molecule, process the first production example with the molecular descriptor generator to produce a first molecular descriptor, pre-process, with the pre-processor, the first molecular descriptor, and process, with the trained chemical super learner model, the pre-processed first molecular descriptor to determine a first prediction for a property of the first molecule.

Embodiments disclosed herein generally relate to a computer-implemented method of training a chemical super learner model that includes obtaining a plurality of training examples from a training database. Each training example includes a simplified molecular-input line-entry system (SMILES) description of a molecule and a first property. The method further includes processing the plurality of training examples, with a molecular descriptor generator to produce a plurality of molecular descriptors and pre-processing, with a pre-processor, the plurality of molecular descriptors. The method further includes training one or more machine-learned models using the pre-processed plurality of molecular descriptors and the training database, wherein each of the one or more machine-learned models are configured to accept a pre-processed molecular descriptor and return a first property prediction. The method further includes scoring the one or more machine learned models, wherein upon scoring each of the one or more machine-learned models has a score and selecting a subset of the one or more machine learned models, wherein each of the machine-learned models in the subset has a better score than the machine-learned models outside of the subset. The method further includes tuning hyperparameters of each of the machine-learned models in the subset, determining a weight for each machine-learned model in the subset, and forming the trained chemical super learner model as a weighted average of each machine-learned model in the subset, wherein each machine-learned model in the subset is weighted in the weighted average according to its weight.

Embodiments disclosed herein generally relate to a non-transitory computer readable medium storing instructions executable by a computer processor, and the instructions include functionality for obtaining a plurality of training examples from a training database. Each training example includes a simplified molecular-input line-entry system (SMILES) description of a molecule and a first property. The instructions further include functionality for processing the plurality of training examples, with a molecular descriptor generator to produce a plurality of molecular descriptors and pre-processing, with a pre-processor, the plurality of molecular descriptors. The instructions further include functionality for training one or more machine-learned models using the pre-processed plurality of molecular descriptors and the training database, wherein each of the one or more machine-learned models are configured to accept a pre-processed molecular descriptor and return a first property prediction and scoring the one or more machine learned models, wherein upon scoring each of the one or more machine-learned models has a score. The instructions further include functionality for selecting a subset of the one or more machine learned models, wherein each of the machine-learned models in the subset has a better score than the machine-learned models outside of the subset, tuning hyperparameters of each of the machine-learned models in the subset, determining a weight for each machine-learned model in the subset, and forming a trained chemical super learner model as a weighted average of each machine-learned model in the subset, wherein each machine-learned model in the subset is weighted in the weighted average according to its weight. The instructions further include functionality for obtaining a first production example from a production database, processing the first production example with the molecular descriptor generator to produce a first molecular descriptor, pre-processing, with the pre-processor, the first molecular descriptor, and processing the trained chemical super learner model to predict the first property for the first production example.

In some embodiments, one or more computer processors are further configured to receive a second production example comprising a SMILES representation of a second molecule, process the second production example with a molecular descriptor generator to produce a second molecular descriptor, pre-process, with a pre-processor, the second molecular descriptor, and process, with a trained chemical super learner model, the pre-processed second molecular descriptor to determine a second prediction for the property of the second molecule.

One or more embodiments disclosed herein include an inversion system configured to, at least, determine a prediction of a property of a molecule by sequentially using a molecular descriptor, pre-processor, and trained chemical super learner model.

In some embodiments, one or more computer processors are further configured to obtain a plurality of production examples from a production database wherein each production example comprises a SMILES representation of a unique molecule, process the plurality of production examples with a molecular descriptor generator to produce a plurality of molecular descriptors, pre-process, with a pre-processor, the plurality of molecular descriptors, and iteratively apply the following steps until stopped by a stopping criterion. The steps include selecting, with an inversion system, a production example from the plurality production examples, and processing, with the trained chemical super learner model, the pre-processed molecular descriptor of the selected production example to determine a selected property prediction. In one or more embodiments the inversion system selects the production example based on the property prediction of all production examples previously processed by the trained chemical super learner model.

In one or more embodiments training examples include a second property.

In one or more embodiments, one or more machine-learned models are trained jointly with a first property and a second property and configured to return a first property prediction and a second property prediction.

In one or more embodiments, a pre-processor is configured by a set of pre-processor parameters.

In one or more embodiments, a molecular descriptor generator accepts a SMILES representation of a molecule for each training example in a plurality of training examples and returns a vector for each training example. In some embodiments, the vector includes a Morgan fingerprint representation of the training example, a Mordred representation of the training example, and an embedding representation of the training example.

In some embodiments, hyperparameters of each of one or more machine-learned models are tuned independently using a genetic algorithm. Further, in one or more embodiments, a weight of each of the machine-learned models is determined using a sequential least-squares programming meta learner.

In one or more embodiments, a generalization error of a trained chemical super learner model is estimated.

In light of the structure and functions described above, embodiments of the invention may include respective means adapted to carry out various steps and functions defined above in accordance with one or more aspects and any one of the embodiments of one or more aspect described herein. Other aspects and advantages of the claimed subject matter will be apparent from the following description and the appended claims.

### BRIEF DESCRIPTION OF DRAWINGS

FIG. 1 depicts a system in accordance with one or more embodiments.

FIG. 2 depicts an example molecular descriptor generator in accordance with one or more embodiments.

FIG. 3 depicts a neural network in accordance with one or more embodiments.

FIG. 4 depicts data processes, transfer, and flow through a trained gradient boosted trees model in accordance with one or more embodiments.

FIG. 5 depicts a flowchart in accordance with one or more embodiments.

FIG. 6A depicts the relative importances of molecular descriptors for a ESOL dataset in accordance with one or more embodiments.

FIG. 6B depicts the relative importances of molecular descriptors for a FreeSolv dataset in accordance with one or more embodiments.

FIG. 6C depicts the relative importances of molecular descriptors for a Lipophilicity dataset in accordance with one or more embodiments.

FIG. 7 depicts a parity plot of measured and predicted yield sooting index (YSI) values for an example chemical super learner model in accordance with one or more embodiments.

FIG. 8A depicts a system in accordance with one or more embodiments.

FIG. 8B depicts a flowchart in accordance with one or more embodiments.

FIG. 9 depicts a flowchart in accordance with one or more embodiments.

FIG. 10 depicts a system in accordance with one or more embodiments.

### DETAILED DESCRIPTION

In the following detailed description of embodiments of the disclosure, numerous specific details are set forth in order to provide a more thorough understanding of the disclosure. However, it will be apparent to one of ordinary skill in the art that the disclosure may be practiced without these specific details. In other instances, well-known features have not been described in detail to avoid unnecessarily complicating the description.

Throughout the application, ordinal numbers (e.g., first, second, third, etc.) may be used as an adjective for an element (i.e., any noun in the application). The use of ordinal numbers is not to imply or create any particular ordering of the elements nor to limit any element to being only a single element unless expressly disclosed, such as using the terms “before,” “after,” “single,” and other such terminology. Rather, the use of ordinal numbers is to distinguish between the elements. By way of an example, a first element is distinct from a second element, and the first element may encompass more than one element and succeed (or precede) the second element in an ordering of elements.

It is to be understood that the singular forms “a,” “an,” and “the” include plural referents unless the context clearly dictates otherwise. Thus, for example, reference to “a parameter” includes reference to one or more of such parameters.

Terms such as “approximately,” “substantially,” etc., mean that the recited characteristic, parameter, or value need not be achieved exactly, but that deviations or variations, including for example, tolerances, measurement error, measurement accuracy limitations and other factors known to those of skill in the art, may occur in amounts that do not preclude the effect the characteristic was intended to provide.

It is to be understood that one or more of the steps shown in the flowcharts may be omitted, repeated, and/or performed in a different order than the order shown. Accordingly, the scope disclosed herein should not be considered limited to the specific arrangement of steps shown in the flowcharts.

Although multiple dependent claims are not introduced, it would be apparent to one of ordinary skill that the subject matter of the dependent claims of one or more embodiments may be combined with other dependent claims, consistent with the disclosure.

Fuels power and support a wide variety of industries and activities, such as, but not limited to: transportation (over land, air, and sea); energy production (e.g., generation of electricity); and heating applications. To meet the ever-increasing energy demands, while reducing negative affects such as emissions, novel fuels with optimized properties must be discovered and produced.

In one aspect, embodiments disclosed herein relate to methods and systems for generating a chemical super learner model, based on one or more machine-learned models, capable of predicting a fuel property. The chemical super learner model works in tandem with a molecular descriptor generator and a pre-processor such that property predictions can be made for a proposed fuel molecule using only the simplified molecular-input line-entry system (SMILES) description of the proposed fuel molecule. Further, the chemical super learner can be used with an inversion system to efficiently identify fuel molecules with high success potential from a database of possible fuels. As such, the chemical space representing possible fuel molecules can be intelligently probed in a high-throughput manner in order to quickly identify novel and next-generation fuels with desired properties.

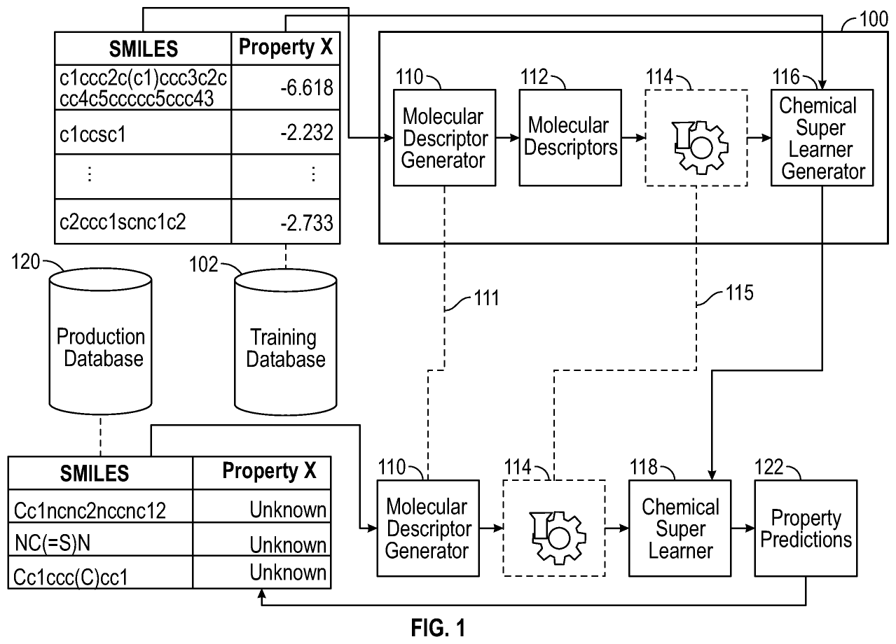

In accordance with one or more embodiments, FIG. 1 depicts a high-level overview of the chemical super learner generation system (100) and a method of use for a generated chemical super learner. As stated, the chemical super learner model described herein is based on one or more machine-learned models. Machine-learning and a more detailed description of the chemical super learner model are provided later in the instant disclosure. For now, it is noted that supervised machine-learned models require examples of input and associated output (i.e., target) pairs in order to learn a desired functional mapping. In one or more embodiments, an input is a simplified molecular-input line-entry system (SMILES) representation of a molecule and the output is a property of the molecule. In one or more embodiments, the property of the molecule is a sensible or latent property or characteristic that can be determined through a measurement of the molecule in bulk or by evaluation of a system or environment of the molecule. That is, while the terms property or molecular property may be used herein, one with ordinary skill in the art will recognize that such a property is not limited to the description, behavior, or characterization of a single, individual molecule. Thus, the terms property and molecular property may be freely applied to a lump of matter made up of the molecule. As an example, consider the molecule known as octane. Using this example, the input associated with octane is the SMILE representation of octane: CCCCCCCC. Further, the associated output (or property) may be the heat of formation, solubility, laminar flame speed, octane number, cetane number, and/or yield sooting index (YSI). As will be described later, in the output of the chemical super learner generated according to the methods and systems described herein may include more than one property.

In accordance with one or more embodiments, and as seen in FIG. 1, multiple input-target pairs are stored in a training database (102). The training database (102) includes, at a minimum, a SMILES representation and at least one associated property for each molecule listed in the training database (102). In FIG. 1, the single property is listed generically as property X. One with ordinary skill in the art will recognize that property X is a placeholder for any molecular property that can be tabulated (e.g., enthalpy, specific heat, YSI, etc.). In one or more embodiments, the training database (102) further includes values for additional properties, which may be generically listed here as property Y (not shown), property Z (not shown), etc. In one or more embodiments, the training database (102) may further include entry items such as a unique identification (ID) number, name, chemical formula, and/or description of each molecule.

Continuing with FIG. 1, the chemical super learner generation system (100) described herein, is configured to receive the SMILES representation and property (property X) of the molecules listed in the training database (102). That is, the chemical super learner generation system (100) is configured to receive data from the training database (102). In one or more embodiments, the chemical super learner generation system (100) includes a molecular descriptor generator (110), a pre-processor (114), and a chemical super learner generator (116). In one or more embodiments, the molecular descriptor generator (110) receives the SMILES representations of the molecules (one or more) in the training database (102) and produces a molecular descriptor (112) for each received molecule SMILES representation. The molecular descriptor (112) for each molecule is a vector (of at least length of one) that provides a numerical representation of the molecule. In one or more embodiments, the molecular descriptor generator (110) uses three numericalization techniques and concatenates the results into a single vector for each received SMILES representation. Specifically, in one or more embodiments, the molecular descriptor generator (110) converts a given SMILES representation of a molecule to a Morgan fingerprint, a set of 2D and 3D descriptor values, and an embedding vector.

The Morgan fingerprint is a reimplementation of the extended connectivity fingerprint (ECFP). Extended connectivity representations create fingerprints of varying lengths and depict circular atom neighborhoods. Generally, a variation accounts for each identifier as often as it appears in the molecule, rather than just once, to keep track of the frequency counts of the ECFP characteristics. This variant is frequently identified as ECFC. Some qualities of ECFPs are they can be calculated quickly, are not pre-defined and represent an infinite variety of different molecular features (including stereochemical information), and have features that indicate the presence of specific substructures, making it easier to understand analysis results. The ECFP algorithm can be modified to generate various circular fingerprints optimized for other applications. In one or more embodiments, the molecular descriptor generator (110) receives a given SMILES representation and produces a bit representation of the molecule known herein as a Morgan fingerprint. The number of bits used in the Morgan fingerprint is determined and supplied by a user. In one or more embodiments, the Morgan fingerprint uses 2048 bits. As such, in one or more embodiments, the molecular descriptor generator (110) further incudes one or more configuration parameters, wherein the configuration parameters dictate the behavior of the molecular descriptor generator (110). For example, the number of bits used by the Morgan fingerprint is a configuration parameter of the molecular descriptor generator (110). In one or more embodiments, the Morgan fingerprint for each molecule in the training database (102) is determined using an open-source software package such as RDKit, which may be readily imported into a Python programming environment.

Descriptor values are numerical values used to quantitatively describe the physical and chemical information of a molecule. A descriptor value must possess the following qualities: 1) invariance concerning labeling and numbering of atoms; 2) invariance to roto-translation; 3) an unambiguous algorithmically computable definition; and 4) values in a suitable numerical range for the set of molecules. Many types of descriptor values exist and can generally be classified as one of: 0D (count descriptors and so on); 1D (list of structural fragments, fingerprints); 2D (graph invariants); 3D (3D-MORSE descriptors, etc.); and 4D descriptor values (Volsurf, etc.). In one or more embodiments, the molecular descriptor generator (110) uses an open-source descriptor value calculation software, referenced herein as the Mordred package, to covert a SMILES representation of molecule to a set of 2D and/or 3D descriptor values. In one or more embodiments, the Mordred package is used to convert each SMILES representation in the training database (102) into a set of 1613 2D descriptor values.

An embedding vector is a numerical description of a molecule inspired by the art of natural language processing (NLP). In one or more embodiments, the molecular descriptor generator (110) uses an open-source embedding package known as Mol2Vec to convert a SMILES into an embedding vector. Mol2Vec molecular substructures derived from the Morgan fingerprinting algorithm as “words” and compounds as “sentences”. Thus, Mol2Vec learns molecular substructure's vector representations that point in similar directions for chemically related substructures. By adding the vectors of the individual substructures, molecules can be described as embedding vectors.

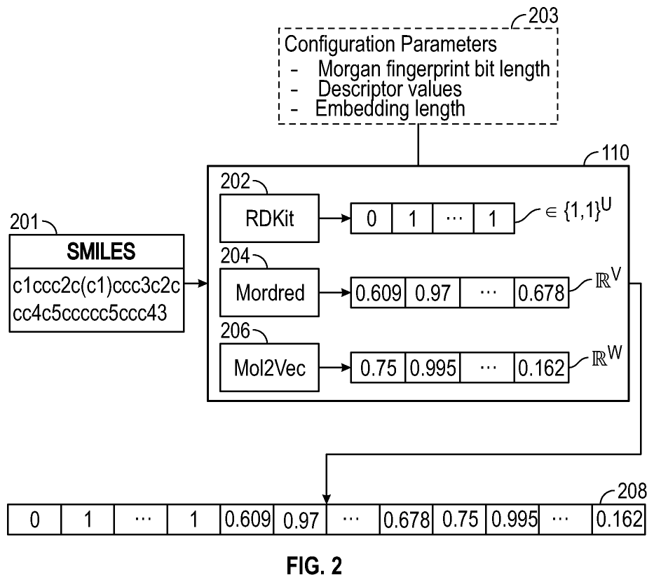

FIG. 2 depicts an example of the molecular descriptor generator (110) applied to a single example SMILES representation (201) of a molecule in accordance with one or more embodiments. As seen in the example of FIG. 2, the molecular descriptor generator (110) accepts a SMILES representation of a molecule, such as the example SMILES representation (201) and processes the SMILES representation with RDKit (202), Mordred (204), and Mol2Vec (206). RDKit (202) processes, or otherwise converts, the example SMILES representation (201) into a Morgan fingerprint representation. In one or more embodiments, the Morgan fingerprint representation is a bit vector of length U, where U is an integer greater than or equal to 1. Mordred (204) processes, or otherwise converts, the example SMILES representation (201) into a vector of descriptor values. In one or more embodiments, the descriptor values output by Mordred (204) are defined by a user-provided set. In other embodiments, the descriptor values are defined by type, for example, 2D descriptor values. Herein, the number of descriptor values output by Mordred (204) for a SMILES representation is V. Mol2Vec processes, or otherwise converts, the example SMILES representation (201) to an embedding vector. The embedding vector has a length of W. In one or more embodiments, the molecular descriptor generator (110) is parameterized by, or has its behavior controlled by, a set of configuration parameters (203). The configuration parameters (203) may define the number of bits used in the Morgan fingerprint, the number or type of descriptor values produced by Mordred (204), and/or the length of the embedding vector. That is, the configuration parameters (203) may directly, or indirectly, affect or define the values of U, V, and W. The molecular descriptor generator (110), for a given SMILES representation, concatenates the output of RDKit (202), Mordred (204), and Mol2Vec (206) to form a molecular descriptor (112) for the associated molecule. For a given molecule, the length of the molecular descriptor (112) output by the molecular descriptor generator (110) is U+V+W. FIG. 2 depicts an example molecular descriptor (208) as output by the molecular descriptor generator (110) upon processing the example SMILES representation (201) according to the configuration parameters (203). In one or more embodiments, the configuration parameters (203) are defined and set by a user. In other embodiments, the configuration parameters (203) are learned while training the machine-learned models of the chemical super learner model. Training of machine-learned models will be described in greater detail later in the instant disclosure.

Returning to FIG. 1, the molecular descriptor generator (110) is used to processes SMILES representations from the training database (102) to produce molecular descriptors (112) (one for each molecule represented in the training database). The molecular descriptors (112) undergo pre-processing by a pre-processor (114). Pre-processing may include identifying molecular descriptors (112) with missing or invalid values, performing an imputation procedure to populate missing or invalid values, or removing elements of molecular descriptor (112) across the entire dataset of molecular descriptors (112). Pre-processing may further include normalizing the molecular descriptors (112). The aforementioned pre-processing step are associated with pre-processing parameters. For example, in the case of normalization, the pre-processing parameters include information about the means and standard deviations (or variances) applied to enact the normalization. Likewise, determined imputation values and identification of elements to be removed are also, if applicable, are also stored as pre-processing parameters. Thus, it may be said that the pre-processor (114) includes, or is configured by, a set of pre-processing parameters. In one or more embodiments, the pre-preprocessing parameters are defined by a user. In other embodiments, the pre-processing parameters are learned while training the machine-learned models of the chemical super learner (to be described in greater detail below).

Upon generating molecular descriptors (112) from the SMILES in the training database (102), and pre-processing the molecular descriptors (112), the pre-processed molecular descriptors (112) are passed with to a chemical super learner generator (116) along with the property, or properties, of the molecules contained in the training database (102). That is, for a given molecule in the training database (102), the chemical super learner generation system (100) forms an input-target pair consisting of the pre-processed molecular descriptor (112) (input) and property (or properties) (target). The input-target pairs are used by the chemical super learner generator (116) to produce a chemical super learner (118). The processes of the chemical super learner generator (116) are described in greater detail later in the instant disclosure. For now, it is sufficient to say that the chemical super learner (118) is configured to accept a pre-processed molecular descriptor (112) of a molecule and output a prediction for one or more properties of the molecule. In the example of FIG. 1, property X is provided to the chemical super learner generator (116) when forming input-target pair for each molecule in the training database (102).

Continuing with FIG. 1, the chemical super learner (118) generated by the chemical super learner generator (116) of the chemical super learner generation system (100) is used in production. The production setting consists of using the chemical super learner (118) to predict the value for the property (or properties) contained in the training database (102) (e.g., property X) for molecules contained in (stored/represented in) a production database (120). The production database (120), similarly to the training database (102), lists (or tabulates) a SMILES representation of at least one molecule. In contrast to the training database (102), the production database (120) does not include property information for any of its listed molecules. In one or more embodiments, the production database (120) may include a null entry, or other placeholder value, for the property (or properties) of its listed molecules. FIG. 1 depicts example contents of a production database (120). As seen in FIG. 1, the example production database (120) contains SMILES representations for three molecules but property X for these molecules is unknown (a placeholder value).

In production, the SMILES representation of one or more molecules listed in the production database (120) are processed with the molecular descriptor generator (110). It is important to note that any configuration parameters (203) specified, or learned, when using the molecular descriptor generator (110) in the chemical super learner generation system (100) are used in the production setting. The sharing of configuration parameters (203) between the chemical super learner generation system (100) and the use of the molecular descriptor generator (110) in the production setting is demonstrated by the first connection (111) in FIG. 1.

As depicted in FIG. 1, upon processing the SMILES representation of one or more molecules in the production database (120) with the molecular descriptor generator (110), the resulting molecular descriptors are pre-processed with the pre-processor (114). As stated, the pre-processor may include various pre-processing parameters defined, or learned, by the chemical super learner generation system (100). These pre-processing parameters are used by the pre-processor (114) when pre-processing the molecules of the production database (120) as indicated by the second connection (115) in FIG. 1.

After pre-processing, the resulting pre-processed molecular descriptors of one or more molecules in the production database (120) are processed by the chemical super learner (118). Recall that the chemical super learner (118) was generated by the chemical super learner generation system (100) operating on the training database (102). The chemical super learner (118) outputs a property prediction for each received pre-processed molecular descriptor. In one or more embodiments, the property predictions (122) are stored in the production database (120) alongside their corresponding molecule. Thus, the chemical super learner (118) can predict a property of a molecule given a pre-processed molecular descriptor of the molecule, where the pre-processed molecular descriptor is determined from only the SMILES representation of the molecule. As such, methods and systems described herein allow for property estimation of molecules, for which the property of the molecules is unknown, given only a SMILES representation of the molecules.

As stated, the chemical super learner model (118) is composed of one or more machine-learned models. Machine learning (ML), broadly defined, is the extraction of patterns and insights from data. The phrases “artificial intelligence.” “machine learning.” “deep learning.” and “pattern recognition” are often convoluted, interchanged, and used synonymously throughout the literature. This ambiguity arises because the field of “extracting patterns and insights from data” was developed simultaneously and disjointedly among a number of classical arts like mathematics, statistics, and computer science. For consistency, the term machine learning (ML), or machine-learned, will be adopted herein, however, one skilled in the art will recognize that the concepts and methods detailed hereafter are not limited by this choice of nomenclature.

Machine-learned model types may include, but are not limited to, k-means, k-nearest neighbors, neural networks, logistic regression, random forests, generalized linear models, and Bayesian regression. Also, machine-learning encompasses model types that may further be categorized as “supervised”, “unsupervised”, “semi-supervised”, or “reinforcement” models. One with ordinary skill in the art will appreciate that additional or alternate machine-learned model categorizations may be defined without departing form the scope of this disclosure. Machine-learned model types are usually associated with additional “hyperparameters” which further describe the model. For example, hyperparameters providing further detail about a neural network may include, but are not limited to, the number of layers in the neural network, choice of activation functions, inclusion of batch normalization layers, and regularization strength. Commonly, in the literature, the selection of hyperparameters surrounding a model is referred to as selecting the model “architecture.”

A brief discussion and summary of various machine-learned model types is provided herein. However, one with ordinary skill in the art will recognize that a full discussion of every type of machine-learned model applicable to the methods and systems disclosed herein is not possible nor required to describe the chemical super learner generation system (100) and use of the resulting chemical super learner model (118). Consequently, the following discussion of machine-learned models is provided by way of introduction to the art of machine-learning and does not impose a limitation on the present disclosure.

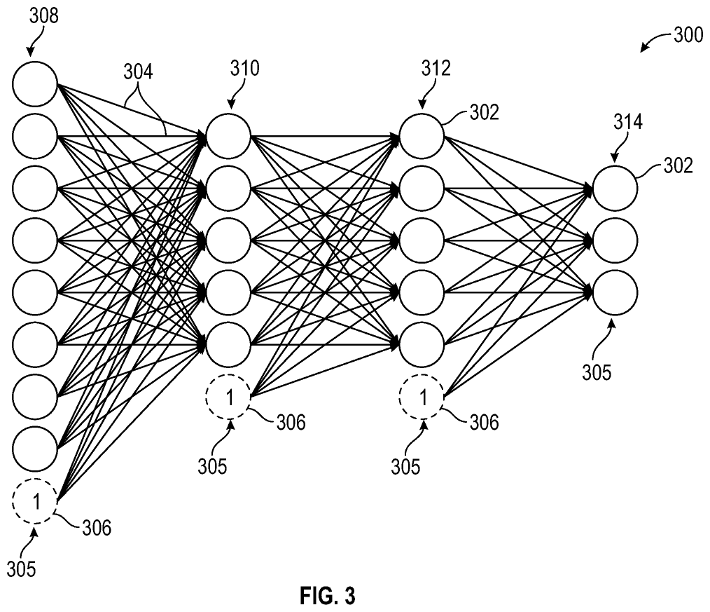

A diagram of a neural network is shown in FIG. 3. At a high level, a neural network (300) may be graphically depicted as being composed of nodes (302), where here any circle represents a node, and edges (304), shown here as directed lines. The nodes (302) may be grouped to form layers (305). FIG. 3 displays four layers (308, 310, 312, 314) of nodes (302) where the nodes (302) are grouped into columns, however, the grouping need not be as shown in FIG. 3. The edges (304) connect the nodes (302). Edges (304) may connect, or not connect, to any node(s) (302) regardless of which layer (305) the node(s) (302) is in. That is, the nodes (302) may be sparsely and residually connected. A neural network (300) will have at least two layers (305), where the first layer (308) is considered the “input layer” and the last layer (314) is the “output layer”. Any intermediate layer (310, 312) is usually described as a “hidden layer”. A neural network (300) may have zero or more hidden layers (310, 312) and a neural network (300) with at least one hidden layer (310, 312) may be described as a “deep” neural network or as a “deep learning method”. In general, a neural network (300) may have more than one node (302) in the output layer (314). In this case the neural network (300) may be referred to as a “multi-target” or “multi-output” network.

Nodes (302) and edges (304) carry additional associations. Namely, every edge is associated with a numerical value. The edge numerical values, or even the edges (304) themselves, are often referred to as “weights” or “parameters”. While training a neural network (300), numerical values are assigned to each edge (304). Additionally, every node (302) is associated with a numerical variable and an activation function. Activation functions are not limited to any functional class, but traditionally follow the form

$$A = f ( {\sum }_{i ∈ ( incoming )} [ {( node value )}_{i} {( edge value )}_{i} ] ) , \qquad ( 1 )$$

where i is an index that spans the set of “incoming” nodes (302) and edges (304) and f is a user-defined function. Incoming nodes (302) are those that, when viewed as a graph (as in FIG. 3), have directed arrows that point to the node (302) where the numerical value is being computed. Some functions for ƒ may include the linear function ƒ(x)=x, sigmoid function

$$f ( x ) = \frac{1}{1 + {e}^{- x}} ,$$

and rectified linear unit function ƒ(x)=max(0,x), however, many additional functions are commonly employed. Every node (302) in a neural network (300) may have a different associated activation function. Often, as a shorthand, activation functions are described by the function ƒ by which it is composed. That is, an activation function composed of a linear function ƒ may simply be referred to as a linear activation function without undue ambiguity.

When the neural network (300) receives an input, the input is propagated through the network according to the activation functions and incoming node (302) values and edge (304) values to compute a value for each node (302). That is, the numerical value for each node (302) may change for each received input. Occasionally, nodes (302) are assigned fixed numerical values, such as the value of 1, that are not affected by the input or altered according to edge (304) values and activation functions. Fixed nodes (302) are often referred to as “biases” or “bias nodes” (306), displayed in FIG. 3 with a dashed circle.

In some implementations, the neural network (300) may contain specialized layers (305), such as a normalization layer, or additional connection procedures, like concatenation. One skilled in the art will appreciate that these alterations do not exceed the scope of this disclosure.

As noted, the training procedure for the neural network (300) comprises assigning values to the edges (304). To begin training, the edges (304) are assigned initial values. These values may be assigned randomly, assigned according to a prescribed distribution, assigned manually, or by some other assignment mechanism. Once edge (304) values have been initialized, the neural network (300) may act as a function, such that it may receive inputs and produce an output. As such, at least one input is propagated through the neural network (300) to produce an output. Training data is provided to the neural network (300). Generally, training data consists of pairs of inputs and associated targets. The targets represent the “ground truth”, or the otherwise desired output, upon processing the inputs. During training, the neural network (300) processes at least one input from the training data and produces at least one output. Each neural network (300) output is compared to its associated input data target. The comparison of the neural network (300) output to the target is typically performed by a so-called “loss function”; although other names for this comparison function such as “error function”, “misfit function”, and “cost function” are commonly employed. Many types of loss functions are available, such as the mean-squared-error function, however, the general characteristic of a loss function is that the loss function provides a numerical evaluation of the similarity between the neural network (300) output and the associated target. The loss function may also be constructed to impose additional constraints on the values assumed by the edges (304), for example, by adding a penalty term, which may be physics-based, or a regularization term. Generally, the goal of a training procedure is to alter the edge (304) values to promote similarity between the neural network (300) output and associated target over the training data. Thus, the loss function is used to guide changes made to the edge (304) values, typically through a process called “backpropagation”.

While a full review of the backpropagation process exceeds the scope of this disclosure, a brief summary is provided. Backpropagation consists of computing the gradient of the loss function over the edge (304) values. The gradient indicates the direction of change in the edge (304) values that results in the greatest change to the loss function. Because the gradient is local to the current edge (304) values, the edge (304) values are typically updated by a “step” in the direction indicated by the gradient. The step size is often referred to as the “learning rate” and need not remain fixed during the training process. Additionally, the step size and direction may be informed by previously seen edge (304) values or previously computed gradients. Such methods for determining the step direction are usually referred to as “momentum” based methods.

Once the edge (304) values have been updated, or altered from their initial values, through a backpropagation step, the neural network (300) will likely produce different outputs. Thus, the procedure of propagating at least one input through the neural network (300), comparing the neural network (300) output with the associated target with a loss function, computing the gradient of the loss function with respect to the edge (304) values, and updating the edge (304) values with a step guided by the gradient, is repeated until a termination criterion is reached. Common termination criteria are: reaching a fixed number of edge (304) updates, otherwise known as an iteration counter; a diminishing learning rate; noting no appreciable change in the loss function between iterations; reaching a specified performance metric as evaluated on the data or a separate hold-out data set. Once the termination criterion is satisfied, and the edge (304) values are no longer intended to be altered, the neural network (300) is said to be “trained.”

Another example of a machine-learned model type is a decision tree. As will be described, decisions trees often act as components, or sub-models, to other types of machine-learned models such as random forests and gradient boosted machines. A decision tree is composed of nodes. A decision is made at each node such that data present at the node are segmented. Typically, at each node, the data at said node, are split into two parts, or segmented bimodally, however, multimodal segmentation is possible. The segmented data can be considered another node and may be further segmented. As such, a decision tree represents a sequence of segmentation rules. The segmentation rule (or decision) at each node is determined by an evaluation process. The evaluation process usually involves calculating which segmentation scheme results in the greatest homogeneity or reduction in variance in the segmented data. However, a detailed description of this evaluation process, or other potential segmentation scheme selection methods, is omitted for brevity and does not limit the scope of the present disclosure.

Further, if at a node in a decision tree, the data are no longer to be segmented, that node is said to be a “leaf node.” Commonly, values of data found within a leaf node are aggregated, or further modeled, such as by a linear model, so that a leaf node represents a class or an aggregated value (e.g., an average). A decision tree can be configured in a variety of ways, such as, but not limited to, choosing the segmentation scheme evaluation process, limiting the number of segmentations, and limiting the number of leaf nodes. Generally, when the number of segmentations or leaf nodes in a decision tree is limited, the decision tree is said to be a “weak learner.”

Commonly, gradient boosted machine models are constructed using decision trees. Hereafter, a gradient boosted machine model using decision trees is referred to as a gradient boosted trees model. In most implementations, the decision trees from which a gradient boosted trees model is composed are weak learners. In a gradient boosted trees model, the decision trees are ensembled in series, wherein each decision tree makes a weighted adjustment to the output of the preceding decision trees in the series. The process of ensembling decision trees in series, and making weighted adjustments, to form a gradient boosted trees model is best illustrated by considering the training process of a gradient boosted trees model.

Training a gradient boosted trees model consists of the selection of segmentation rules for each node in each decision tree; that is, training each decision tree. Once trained, a decision tree is capable of processing data. For example, a decision tree may receive a data input (e.g., a pre-processed molecular descriptor). The data input is sequentially transferred to nodes within the decision tree according to the segmentation rules of the decision tree. Once the data input is transferred to a leaf node, the decision tree outputs the assigned class or aggregate value (e.g., molecular property value) of the associated leaf node.

Generally, training a gradient boosted model firstly consists of making a simple prediction (SP) for the target data (i.e., the one or more molecular properties). The simple prediction (SP) may be the average property value over the training database (102). The simple prediction (SP) is subtracted from the targets to form a first residuals. The first decision tree in the series is created and trained, wherein the first decision tree attempts to predict the first residuals forming first residual predictions. The first residual predictions from the first decision tree are scaled by a scaling parameter. In the context of gradient boosted trees the scaling parameter is known as the “learning rate” (η). The learning rate is one of the hyperparameters governing the behavior of the gradient boosted trees model. The learning rate (η) may be fixed for all decision trees or may be variable or adaptive. The first residual predictions of the first decision tree are multiplied by the learning rate (η) and added to the simple prediction (SP) to form a first predictions. The first predictions are subtracted from the targets to form a second residuals. A second decision tree is created and trained using the data inputs and the second residuals as targets such that it produces second residual predictions. The second residual predictions are multiplied by the learning rate (η) and are added to the first predictions forming second predictions. This process is repeated recursively until a termination criterion is achieved.

Many termination criteria exist and are not all enumerated here for brevity. Common termination criteria are terminating training when a pre-defined number of decision trees has been reached, or when improvement in the residuals is no longer observed.

Once trained, a gradient boosted trees model may make predictions using input data. To do so, the input data is passed to each decision tree, which will form a plurality of residual predictions. The plurality of residual predictions is multiplied by the learning rate (η), summed across every decision tree, and added to the simple prediction (SP) formed during training to produce the gradient boosted trees predictions.

One with ordinary skill in the art will appreciate that many adaptions may be made to gradient boosted trees models and that these adaptions do not exceed the scope of this disclosure. Some adaptions may be algorithmic optimizations, efficient handling of sparse data, use of out-of-core computing, and parallelization for distributed computing. Commonly, when such adaptions are applied to a gradient boosted trees model, the model is known in the literature as XGBoost.

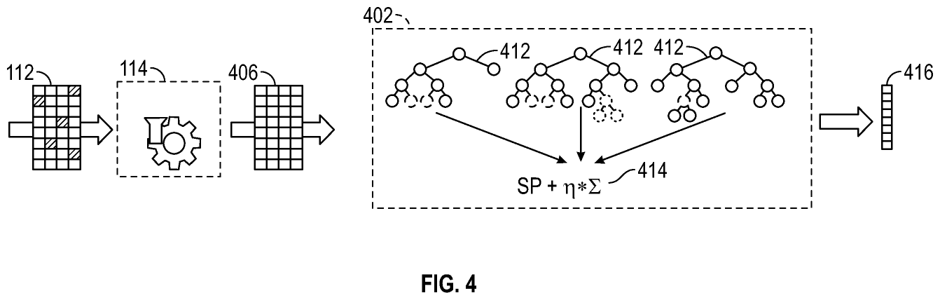

FIG. 4 depicts, generally, the flow of data through a trained gradient boosted trees model (402) in accordance with one or more embodiments. For concision, in FIG. 4, it is assumed that one or more SMILES representations have been previously processed by the molecular descriptor generator (110). Thus, the processes of FIG. 4 begin with a set of one or more molecular descriptors (112) as input data to the gradient boosted trees model (402). The molecular descriptors (112) are pre-processed by the pre-processor (114) as previously described. The result of the pre-processing is pre-processed molecular descriptors (406).

The pre-processed molecular descriptors (406) are passed to the gradient boosted trees model (402) composed of a plurality of decision trees (412). As such, the pre-processed molecular descriptors (406) are processed by each decision tree (412) and the output of each decision tree is collected, multiplied by the learning rate (η), summed, and added to the simple prediction (SP) established during training forming an ensemble (414). The result of the ensemble (414) is returned as the gradient boosted trees model prediction (416). In the context of the current disclosure, the gradient boosted trees model prediction (416) is the property predictions (122).

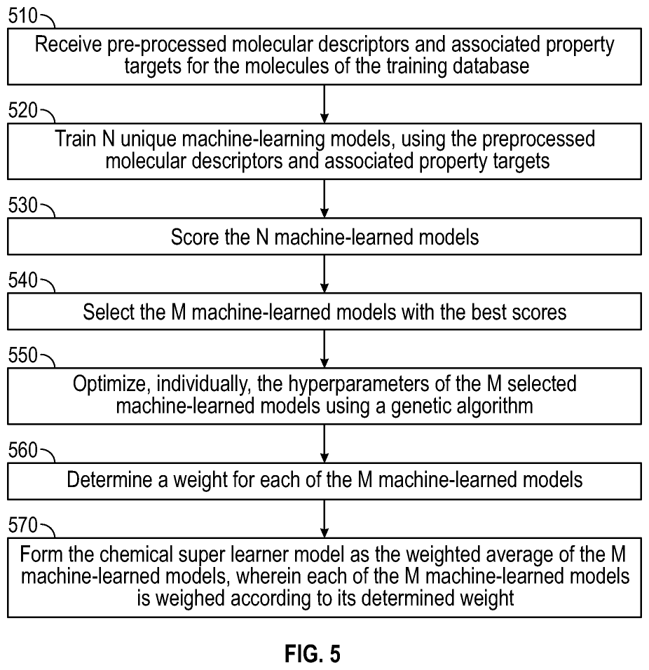

With an introduction to machine-learning and some machine-learned model types presented, the processes of the chemical super learner generator (116) are described in greater detail here. Turning to FIG. 5, FIG. 5 depicts a flowchart outlining the processes of the chemical super learner generator (116) in accordance with one or more embodiments. As depicted in FIG. 5, in Block 510 the chemical super learner generator (116) receives pre-processed molecular descriptors and associated target properties for the molecules of the training database (102). Each molecule may be associated with one or more properties. Further, each molecule in the database is represented with a SMILES representation that is processed by the molecular descriptor generator (110) and pre-processor (114) to produce the pre-processed molecular descriptors.

In one or more embodiments, the pre-processed molecular descriptors are split into various sets such as a training set, validation set, and testing set for training, scoring, and generalization error estimation purposes. Because data splitting is a common practice when training, evaluating, and testing a machine-learned model, it is not explicitly depicted in FIG. 5. One with ordinary skill in the art will recognize that any data splitting technique may be applied to the pre-processed molecular descriptors without departing from the scope of this disclosure. In one or more embodiments, the molecules of the training database (102) are split using a molecular scaffold splitting technique such that the molecules in any of the resulting data partitions are structurally dissimilar. In one or more embodiments, the molecules of the training database (102) are split into one or more datasets before being processed by the molecular descriptor generator (110) and/or pre-processor (114) such that the configuration parameters and/or pre-processor parameters may be learned in training according to a training dataset. Thus, it is understood that training, evaluation, and optimization an any machine-learned models discussed below may be according to one or more pre-defined partitions of the training database (102).

In Block 520, N unique machine-learned models are trained independently using the pre-processed molecular descriptors and associated property targets, where N is an integer greater than or equal to 1. In one or more embodiments, the N machine-learned models are trained using a cross-validation scheme applied to a training dataset (or partition) of the pre-processed molecular descriptors. In one or more embodiments, 40 machine-learned models are trained, where the type, sub-type, and shorthand name of each model is represented in Table I.

TABLE I
The type, subtype, model, and shorthand model name of 40 machine-learned models
trained using pre-processed molecular descriptors and target properties in
accordance with one or more embodiments of the instant disclosure.

| | | | | Model |
|---|---|---|---|---|
| Type | Sub-type | S No | Model | name |
| Linear models | Classical | 1 | LinearRegression | LR |
| | | 2 | Ridge | RR |
| | | 3 | RidgeCV | RCVR |
| | | 4 | SGDRegressor | SGDR |
| | Regressors | 5 | ElasticNet | ENR |
| | with variable | 6 | ElasticNetCV | ENCVR |
| | selection | 7 | Lars | LaR |
| | | 8 | LarsCV | LaCVR |
| | | 9 | Lasso | LasR |
| | | 10 | LassoCV | LasCVR |
| | | 11 | LassoLars | LLR |
| | | 12 | LassoLarsIC | LLICR |
| | | 13 | LassoLarsCV | LLCVR |
| | | 14 | OrthogonalMatchingPursuit | OMPR |
| | | 15 | OrthogonalMatchingPursuitCV | OMPCVR |
| | Bayesian | 16 | BayesianRidge | BRR |
| | regressors | | | |
| | Outlier-robust | 17 | RANSACRegressor | RANR |
| | regressors | 18 | HuberRegressor | HR |
| | Generalized | 19 | PoissonRegressor | PR |
| | linear models | 20 | GammaRegressor | GR |
| | (GLM) for | 21 | TweedieRegressor | TR |
| | regression | | | |
| | Miscellaneous | 22 | PassiveAggressiveRegressor | PAR |
| Kernel Ridge models | Kernel Ridge | 23 | KernelRidge | KRR |
| | Regression | | | |
| Gaussian Processes | Gaussian | 24 | GaussianProcessRegressor | GPR |
| | Processes | | | |
| Nearest Neighbors | Nearest | 25 | KNeighborsRegressor | KNR |
| | Neighbors | | | |
| Neural network models | Neural network | 26 | MLPRegressor | MLPR |
| | models | | | |
| Support Vector Machines | Support Vector | 27 | SVR | SVR |
| | Machines | 28 | LinearSVR | LSVR |
| | | 29 | NuSVR | NSVR |
| Decision Trees | Classical | 30 | DecisionTreeRegressor | DTR |
| | | 31 | ExtraTreeRegressor | ETR |
| | Ensemble | 32 | ExtraTreesRegressor | ETsR |
| | Methods | 33 | RandomForestRegressor | RFR |
| | | 34 | AdaBoostRegressor | ABR |
| | | 35 | BaggingRegressor | BRR |
| | | 36 | GradientBoostingRegressor | GBR |
| | | 37 | HistGradientBoostingRegressor | HGBR |
| | External | 38 | XGBRegressor | XGBR |
| | | 39 | LGBMRegressor | LGBMR |
| | | 40 | CatBoostRegressor | CBR |

Continuing, with FIG. 5, once trained, the N machine-learned models are scored. A model's score represents the ability of the model to accurately predict the target property of a molecule given the pre-processed molecular descriptor of the molecule. Thus, the score of the model is computed by comparing the property predictions produced by the model to the true (known) property targets. Any scoring or comparison function known in the art may be used, including but not limited to: mean absolute error, mean square error, root mean square error, R², etc. Further, scoring may take place using a cross-validation scheme or across a partition of the training database (102) not used during training such as a validation dataset and/or test dataset.

Once scored, in Block 540 the M top scoring machine-learned models are selected for further use, where M is an integer and 1≤M≤N. In Block 550 the hyperparameters of each of the M selected machine-learned models are optimized. The hyperparameters of the M models are optimized independent from each other. In one or more embodiments, the hyperparameter optimization is guided by an evaluation score determined using a cross-validation scheme over a training dataset. In other embodiments, the hyperparameter optimization is guided by an evaluation score over a validation set. In accordance with one or more embodiments, the hyperparameter optimization is performed using a genetic algorithm. It is noted that the order of Blocks 540 and 550 may be readily interchanged without departing from the scope of this disclosure. That is, in some embodiments, the hyperparameters of all N machine-learned models are optimized (or tuned) and then the top M performing models are selected. However, the embodiment depicted in FIG. 5 is generally preferred as hyperparameter optimization can be a computationally expensive task such that it is beneficial to only optimized the hyperparameters of M machine-learned models where M≤N.

After hyperparameter optimization the M selected models are combined to form the chemical super learner model (118). The combination is performed according to a weighted average. Specifically, if ψ represents one of the M selected machine-learned models, then the chemical super learner model (118), Ψ_{SL}, is given by

$${Ψ}_{S L} = {\sum }_{m = 1}^{M} {w}_{m} {ψ}_{m} , {w}_{m} \geq 0 , \qquad ( 2 )$$

where wₘ represents the weight associated with the m^{th} machine-learned model. To ensure that the M selected machine-learned models are combined according to a proper weighted average, EQ. 2 is subject to the following constraint,

$${\sum }_{m = 1}^{M} {w}_{m} = 1 . \qquad ( 3 )$$

Thus, to form the chemical super learner model (118), for each of the M selected machine-learned models, a weight wₘ must be determined. In Block 560, a weight is determined for each of the M machine-learned models. In one or more embodiments, the weights are determined using a Sequential Least-Squares Programming method (SLSQP) to include the equality constraint of EQ. 3. Specifically, the SLSQP method determines the weights for the chemical super learner model (118), Ψ_{SL}, that result in the highest performing (i.e., scoring) model when evaluated on a validation set and/or test set of the molecules of the training database (102). Once the weights of the chemical super learner model (118) have been determined according to Block 560, the chemical super learner model (118) is officially formed as the weighted average of the M selected machine-learned models (EQ. 2) as depicted in Block 570.

While the various blocks in FIG. 5 are presented and described sequentially, one of ordinary skill in the art will appreciate that some or all of the blocks may be executed in different orders, may be combined or omitted, and some or all of the blocks may be executed in parallel. Furthermore, the blocks may be performed actively or passively.

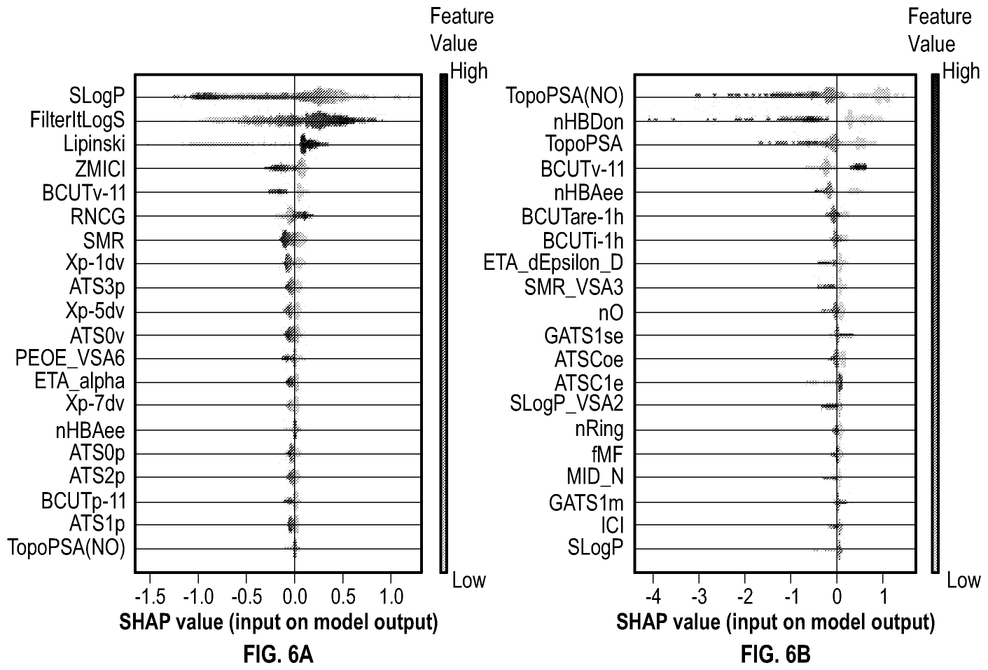

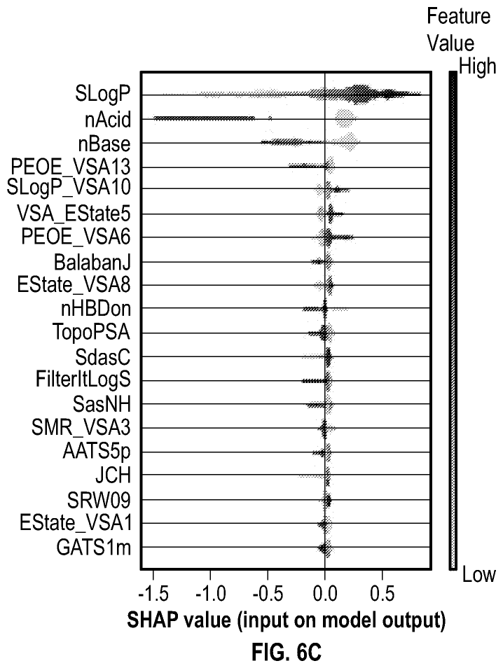

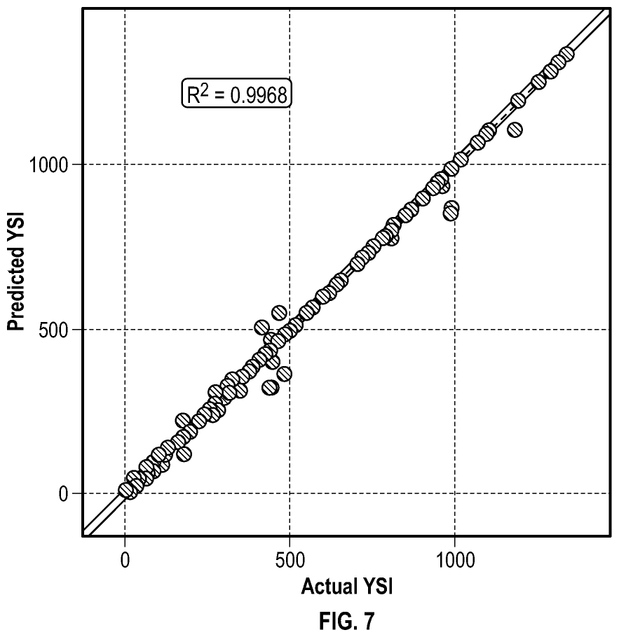

As a concrete example of the methods, processes, models, and techniques described herein, a plurality of synthetic datasets was generated, the chemical super learner generation system (100) was applied independently to four training databases, where each training database included SMILES representations of multiple molecules and a single associated property. These results are shown in FIGS. 6-7 and TABLES II-IV with an accompanying discussion herein.

In accordance with one or more embodiments, the chemical super learner generation system (100) was applied to a training database described herein as the ESOL dataset. At the time of writing, the ESOL dataset contains the SMILES representations of 1127 molecules and the property of the dataset is water solubility. In this example, the molecular descriptor generator (100) produced a Morgan fingerprint (RDKit), Mordred-generated 2D descriptor values, and an embedding vector (Mol2Vec) for each of the 1127 molecules from the SMILES representations. The Morgan fingerprint, 2D descriptor values, and embedding vector were concatenated to form a molecular descriptor for each molecule. The pre-processor (114) was configured to remove elements from the molecular descriptors if the element was not available for each of the 1127 molecules. Upon pre-processing, each molecular descriptor contained 1060 elements. In this example, the ESOL dataset was randomly partitioned into a training dataset and test dataset according to a 4:1 ratio. The random partition was performed 5 times so that the performance of the resulting chemical super learner model (118) could be evaluated and averaged over 5 independent runs. The 40 machine-learned models listed in TABLE I were trained and the top 3 scoring machine-learned models were selected, where the scoring function was the root mean squared error (RMSE) function. The three top performing machine-learned models were, referencing their shorthand names: LLCVR, ETsR, and CBR. After tuning the hyperparameters of these machine-learned models (with a genetic algorithm), their weights were determined to create the chemical super learner model (118). Specifically, the optimal weight the selected machine-learned models was as follows: LLCR: 0.10; ETsR: 0.06; and CBR: 0.84. Thus, using the methods and systems described herein, a chemical super learner model (118) capable of predicting the water solubility of a molecule given a set of molecular descriptors (where the molecular descriptors are formed from a SMILES representation) was generated. The performance of the formed chemical super learner model (118), measured using RMSE, compared to other models in the literature applied to the same dataset (ESOL) is demonstrated in TABLE II. Because multiple runs are considered, the average and standard deviation of the RMSE over the test set are listed in TABLE II, where applicable. As seen, the chemical super learner model outperforms all prior models and results described in the literature concretely demonstrating an advantage of the instant disclosure.

TABLE II
Performance results of the chemical super learner model developed for
the ESOL dataset compared to other models known in the literature.

| Year | | 2017 | 2017 | 2017 | 2017 | 2019 | 2019 | 2019 | 2019 | 2021 | 2022 |
|---|---|---|---|---|---|---|---|---|---|---|---|
| ML Model | | GC^{a} | Weave^{a} | DAG^{a} | MPNN^{a} | Attentive | D-MPNN^{b} | PAGTN^{b} | EIGNN^{a} | SVM/SVM/ | Chemical |
| | | | | | | FP^{a} | | | | Attentive | Super |
| | | | | | | | | | | FP^{a} | Learner |
| References | | Wu | Wu | Wu | Wu | Xiong | Yang | Chen | Chen | Jiang | Current |
| | | et al. | et al. | et al. | et al. | et al. | et al. | et al. | et al. | et al. | study |
| ESOL | Mean | 0.970 | 0.610 | 0.820 | 0.580 | 0.503 | 0.665 | 0.554 | 0.653 | 0.569 | 0.386 |
| | Std | 0.010 | 0.070 | 0.080 | 0.030 | 0.076 | 0.052 | 0.060 | 0.025 | 0.052 | 0.018 |

In accordance with one or more embodiments, the chemical super learner generation system (100) was applied to a training database described herein as the FreeSolv dataset. At the time of writing, the FreeSolv dataset contains the SMILES representations of 639 molecules and the property of the dataset is hydration free energy. In this example, the molecular descriptor generator (100) produced a Morgan fingerprint (RDKit), Mordred-generated 2D descriptor values, and an embedding vector (Mol2Vec) for each of the 639 molecules from the SMILES representations. The Morgan fingerprint, 2D descriptor values, and embedding vector were concatenated to form a molecular descriptor for each molecule. The pre-processor (114) was configured to remove elements from the molecular descriptors if the element was not available for each of the 639 molecules. Upon pre-processing, each molecular descriptor contained 1060 elements. In this example, the FreeSolv dataset was randomly partitioned into a training dataset and test dataset according to a 4:1 ratio. The random partition was performed 5 times so that the performance of the resulting chemical super learner model (118) could be evaluated and averaged over 5 independent runs. The 40 machine-learned models listed in TABLE I were trained and the top 3 scoring machine-learned models were selected, where the scoring function was the root mean squared error (RMSE) function. The three top performing machine-learned models were, referencing their shorthand names: RCVR, ETsR, and CBR. After tuning the hyperparameters of these machine-learned models (with a genetic algorithm), their weights were determined to create the chemical super learner model (118). Specifically, the optimal weight the selected machine-learned models was as follows: RCVR: 0.24; ETsR: 0.50; and CBR: 0.26. Thus, using the methods and systems described herein, a chemical super learner model (118) capable of predicting the hydration free energy of a molecule given a set of molecular descriptors (where the molecular descriptors are formed from a SMILES representation) was generated. The performance of the formed chemical super learner model (118), measured using RMSE, compared to other models in the literature applied to the same dataset (FreeSolv) is demonstrated in TABLE III. Because multiple runs are considered, the average and standard deviation of the RMSE over the test set are listed in TABLE III, where applicable. As seen, the chemical super learner model outperforms all prior models and results described in the literature concretely demonstrating an advantage of the instant disclosure.

TABLE III
Performance results of the chemical super learner model developed for
the FreeSolv dataset compared to other models known in the literature.

| Year | | 2017 | 2017 | 2017 | 2017 | 2017 | 2018 | 2019 | 2019 | 2019 | 2021 | 2022 |
|---|---|---|---|---|---|---|---|---|---|---|---|---|
| ML Model | | GC^{a} | Weave^{a} | DAG^{a} | MPNN^{a} | GCN^{a} | EAGCN^{a} | Attentive | D- | EIGNN^{a} | SVM/SVM/ | Chemical |
| | | | | | | | | FP^{a} | MPNN^{b} | | Attentive | Super |
| | | | | | | | | | | | FP^{a} | Learner |
| References | | Wu | Wu | Wu | Wu | Li | Shang | Xiong | Yang | Chen | Jiang | Current |
| | | et al. | et al. | et al. | et al. | et al. | et al. | et al. | et al. | et al. | et al. | study |
| FreeSolv | Mean | 1.400 | 1.220 | 1.630 | 1.150 | 1.120 | 0.950 | 0.736 | 1.167 | 1.273 | 0.852 | 0.561 |
| | Std | 0.160 | 0.280 | 0.180 | 0.120 | | 0.140 | 0.037 | 0.150 | 0.137 | 0.171 | 0.076 |

In accordance with one or more embodiments, the chemical super learner generation system (100) was applied to a training database described herein as the Lipophilicity dataset. At the time of writing, the Lipophilicity dataset contains the SMILES representations of 4200 molecules and the property of the dataset is the octanol/water distribution coefficient. In this example, the molecular descriptor generator (100) produced a Morgan fingerprint (RDKit), Mordred-generated 2D descriptor values, and an embedding vector (Mol2Vec) for each of the 4200 molecules from the SMILES representations. The Morgan fingerprint, 2D descriptor values, and embedding vector were concatenated to form a molecular descriptor for each molecule. The pre-processor (114) was configured to remove elements from the molecular descriptors if the element was not available for each of the 4200 molecules. Upon pre-processing, each molecular descriptor contained 997 elements. In this example, the Lipophilicity dataset was randomly partitioned into a training dataset and test dataset according to a 4:1 ratio. The random partition was performed 5 times so that the performance of the resulting chemical super learner model (118) could be evaluated and averaged over 5 independent runs. The 40 machine-learned models listed in TABLE I were trained and the top 3 scoring machine-learned models were selected, where the scoring function was the root mean squared error (RMSE) function. The three top performing machine-learned models were, referencing their shorthand names: HGBR, ETsR, and CBR. After tuning the hyperparameters of these machine-learned models (with a genetic algorithm), their weights were determined to create the chemical super learner model (118). Specifically, the optimal weight the selected machine-learned models was as follows: HGBR: 0.28; ETsR: 0.17; and CBR: 0.55. Thus, using the methods and systems described herein, a chemical super learner model (118) capable of predicting the octanol/water distribution coefficient of a molecule given a set of molecular descriptors (where the molecular descriptors are formed from a SMILES representation) was generated. The performance of the formed chemical super learner model (118), measured using RMSE, compared to other models in the literature applied to the same dataset (Lipophilicity) is demonstrated in TABLE IV. Because multiple runs are considered, the average and standard deviation of the RMSE over the test set are listed in TABLE IV, where applicable. As seen, the chemical super learner model outperforms all prior models and results described in the literature concretely demonstrating an advantage of the instant disclosure.

TABLE IV
Performance results of the chemical super learner model developed for the Lipophilicity dataset compared to other models known in the literature.

| Year | | 2017 | 2017 | 2017 | 2017 | 2018 | 2019 | 2019 | 2019 | 2019 | 2021 | 2022 |
|---|---|---|---|---|---|---|---|---|---|---|---|---|
| ML Model | | GC^{a} | Weave^{a} | DAG^{a} | MPNN^{a} | EAGCN^{a} | Attentive | D- | PAGTN^{b} | EIGNN^{a} | SVM/SVM/ | Chemical |
| | | | | | | | FP^{a} | MPNN^{b} | | | Attentive | Super |
| | | | | | | | | | | | FP^{a} | Learner |
| References | | Wu | Wu | Wu | Wu | Shang | Xiong | Yang | Chen | Chen | Jiang | Current |
| | | et al. | et al. | et al. | et al. | et al. | et al. | et al. | et al. | et al. | et al. | study |
| Lipophilicity | Mean | 0.655 | 0.715 | 0.835 | 0.719 | 0.610 | 0.578 | 0.596 | 0.596 | 0.776 | 0.553 | 0.468 |
| | Std | 0.036 | 0.035 | 0.039 | 0.031 | 0.020 | 0.018 | 0.050 | 0.050 | 0.071 | 0.035 | 0.009 |

As depicted in TABLES II-IV, the chemical super learner model (118) generated with methods and systems disclosed herein results in a marked improvement over all other known and state-of-the-art models. An additional advantage of methods and systems of the instant disclosure is that the chemical super learner model (118), once trained, can be evaluated using feature importance methods, such as SHAP analysis, to identify the elements (or features) of the molecular descriptors with the greatest predictive power on a property of interest (e.g., hydration free energy, water solubility, etc.). FIGS. 6A-6C depict the SHAP analysis of the chemical super learner models developed for the ESOL, FreeSolv, and Lipophilicity datasets, respectively. Using feature importance methods the chemical super learner models may be interpreted and given a physical explanation by subject matter experts and researchers.

As a final example, the chemical super learner generation system (100) was applied to a yield sooting index (YSI) database consisting of the SMILES representations and YSI measurements for 446 molecules. Yield sooting index (YSI) is a critical property of fuels that quantifies the fuel tendency to form soot and is based on the measurement of a maximum soot volume fraction that is directly proportional to sooting propensity. The tendency of a pure component or a mixture to produce soot in a diffusion flame is attributed to the sooting propensity measured by YSI values. Thus, YSI data is valuable for predicting the emissions of proposed new fuels. Testing the 40 machine-learned models listed in TABLE I, in this example, the top 3 machine-learned models were CBR, HGBR, and GBR. Specifically, the weights of these machine-learned models in the generated chemical super learner model were determined to be: CBR: 0.89; GBR: 0.08; and HGBR: 0.03. FIG. 7 depicts a parity plot of measured and predicted YSI values (R2=0.9968) using the generated chemical super learner model (118). As seen in FIG. 7, the predictive performance the generated super learner model (118) on the YSI dataset is excellent.

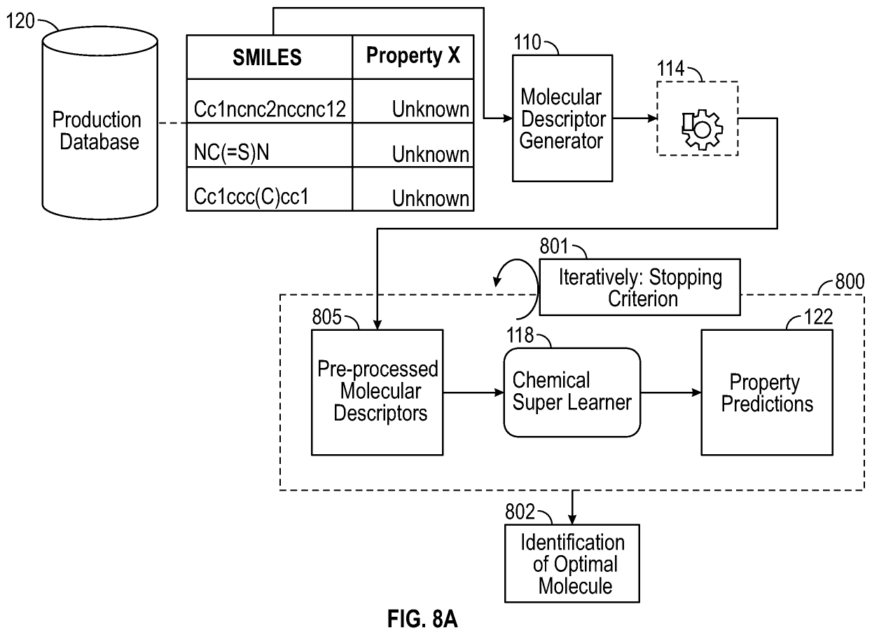

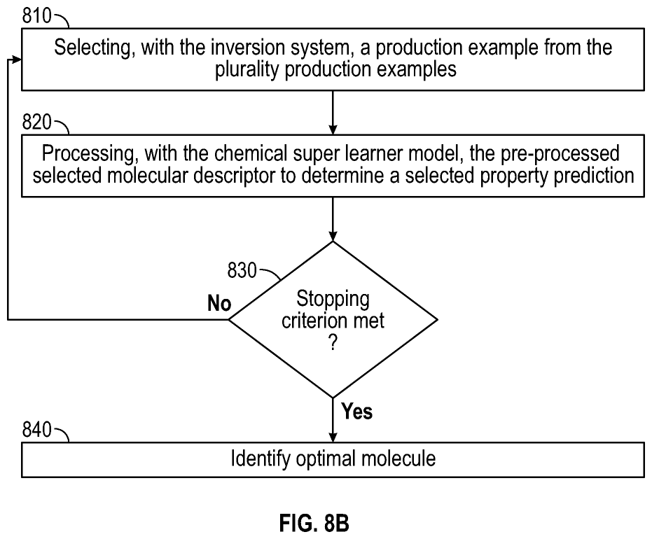

In accordance with one or more embodiments, a generated chemical super learner model (118) is used in an inversion system to quickly identify one or more molecules with optimal property values. An example of the inversion process is shown in FIG. 8A. As seen in FIG. 8A, a production database (120) including SMILES representations of one or more molecules with an unknown value for property X is provided. A previously generated chemical super learner model (118), configured to make a prediction on property X, is provided to the inversion system (800). In one or more embodiments, the SMILES representations of the molecules in the production database (120) are processed by the molecular descriptor generator (110) and pre-processor (114) to create a set of pre-processed molecular descriptors (805), one for each molecule in the production database (120). The molecular descriptor generator (110) and pre-processor (114) use the same configuration parameters and pre-processor parameters defined, or learned, while generating the chemical super learner model (118). In one or more embodiments, the purpose of the inversion system (800) is to identify one or more molecules in the production database (120) that have an optimal value for property X without needing to process every molecule in the production database (120). That is, the inversion system (800) intelligently probes and explores the production database for a molecule with an optimal value for property X. In one or more embodiments, the inversion system (800) can identify similar and/or related molecules by comparison of their pre-processed molecular descriptors (805). In one or more embodiments, the strength of the relationship between two molecules is given as the inverse of the L2 norm of the difference vector between pre-processed molecular descriptors (805) of the molecules. In other embodiments, the strength of the relationship between two molecules is further weighted according to the elements of the pre-processed molecular descriptors (805), where the weights are given by the feature importances of the chemical super learner model (118).

One with ordinary skill in the art will recognize that other methods, known in the art, may be readily applied to identify and rank relationships between molecules in the production database (120) using the pre-processed molecular descriptors (805) without departing from the scope of this disclosure. For example, in one or more embodiments a clustering algorithm is applied to the pre-processed molecular descriptors (805).

Continuing with FIG. 8A, in one or more embodiments, the inversion system (800) iteratively selects a batch of pre-processed molecular descriptors (805) and processes the batch with the chemical super learner model (118) to make a prediction on property X for each molecule in the batch until a stopping criterion (801) is reached. The number of molecules in a batch must be at least one and cannot exceed the number of molecules listed in the production database (120). In each iteration, the inversion system (800) checks the property predictions (122) for the batch and, if a new iteration is required (i.e., stopping criterion is not met) uses the property predictions (122) of the processed batch to intelligently identify and select a new batch of pre-processed molecular descriptors (molecules). That is, the inversion system (800) uses the known prediction results to select new molecules for processing with the chemical super learner model (118). In one or more embodiments, the stopping criterion (801) includes a max iteration counter. That is, the inversion system (800) counts the number of iterations, or batches of molecules processed, and when the number of iterations reaches a pre-defined max iteration limit, the stopping criterion (801) is triggered. In other embodiments, the stopping criterion (801) includes a threshold value for the property X. That is, if a property prediction (122) for one or more molecules in a processed batch exceeds a pre-defined threshold, then these molecules are considered to have optimal property values and the stopping criterion (801) is triggered. In one or more embodiments, the output of the inversion system (800) is an identification of the optimal molecule (802). Again, it is emphasized that not all molecules in the production database (120) need to be processed by the chemical super learner (118). In one or more embodiments, the inversion system (800) is based on a Bayesian search method.

FIG. 8B outlines the processes of the inversion system (800), in accordance with one or more embodiments. In Block 810, the inversion system (800) selects a production example from a plurality of production examples. The plurality of production examples are contained in the production database (120) and each production example includes, at least, a SMILES representation of a molecule and a property value for that molecule. In one or more embodiments, the inversion system (800) may select more than one production example in Block 810. In Block 820, the selected production example is processed by the chemical super learner model, where it is well understood that the production example may be converted to a pre-processed molecular descriptor. In Block 830, the stopping criterion (801) of the inversion system (800) is checked. If the stopping criterion (801) is not met, then the inversion system (800) returns to Block 810 to select a new production example. The inversion system (800) uses the results all previously processed production examples to select a new production example. If the stopping criterion (801) is met, then in Block 840 the optimal molecule is identified and returned to a user.

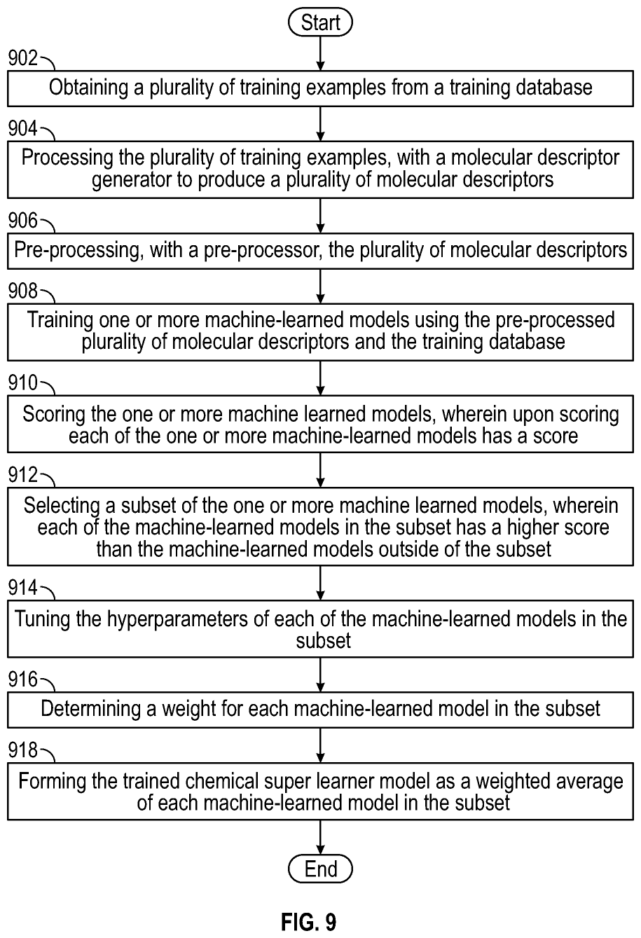

FIG. 9 depicts a flowchart outlining the processes of developing the chemical super learner model (118) in accordance with one or more embodiments. In Block 902, a training database (102) is received. The training database contains a plurality of training examples where each training example includes, at least, a SMILES representation of a molecule and a property of the molecule. In Block 904, the plurality of training examples are processed with a molecular descriptor generator (110) to form a plurality of molecular descriptors. In Block 906, the plurality of molecular descriptors is pre-processed with a pre-processor (114). In one or more embodiments, the molecular descriptor generator (110) and the pre-processor (114) are associated with, or configured with, a set of configuration parameters and pre-processing parameters, respectively. In Block 908, one or more machine-learned models are trained using the pre-processed plurality of molecular descriptors and the training database. That is, a pre-processed molecular descriptor and associated property value form an input-target pair for training the one or more machine-learned models. In Block 910, the one or more machine-learned models are scored. The scoring uses a scoring function that compares the predictions of a trained machine-learned model to the correct targets. In one or more embodiments, the root mean square error (RMSE) function is used to score the one or more machine-learned models. In Block 912, a subset of the one or more machine-learned models is selected. The selection is such that the machine-learned models of the subset have better scores than the unselected machine-learned models of the one or more machine-learned models. In Block 914, the hyperparameters of the machine-learned models in the subset are tuned. In one or more embodiments, the hyperparameter tuning is performed with a genetic algorithm. In one or more embodiments, Block 914 occurs before Block 912. In Block 916 a weight for each machine-learned model in the subset is determined. In one or more embodiments, the weights are determined using an SLSQP meta learner. In Block 918, a chemical super learner model is formed as a weighted average of the machine-learned models in the subset according to the weights determined in Block 916.

In one or more embodiments, the chemical super learner model is used with an inversion system (800) to identify one or more molecules with desired molecular properties (or property). In one or more embodiments, identified molecules are synthesized and characterized in a laboratory setting. In one or more embodiments, identified molecules that pass tests performed in a laboratory setting are fielded and used in real life settings such as transportation and energy equipment and sectors.

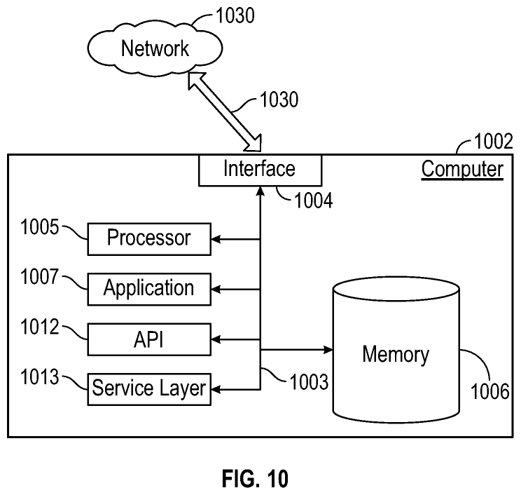

FIG. 10 shows a system in accordance with one or more embodiments. FIG. 10 depicts a block diagram of the computer system (1002) used to provide computational functionalities associated with described algorithms, methods, functions, processes, flows, and procedures as described in this disclosure, according to one or more embodiments. The illustrated computer (1002) is intended to encompass any computing device such as a server, desktop computer, laptop/notebook computer, wireless data port, smart phone, personal data assistant (PDA), tablet computing device, one or more processors within these devices, or any other suitable processing device, including both physical or virtual instances (or both) of the computing device. Additionally, the computer (1002) may include a computer that includes an input device, such as a keypad, keyboard, touch screen, or other device that can accept user information, and an output device that conveys information associated with the operation of the computer (1002), including digital data, visual, or audio information (or a combination of information), or a Graphical User Interface (GUI).

The computer (1002) can serve in a role as a client, network component, a server, a database or other persistency, or any other component (or a combination of roles) of a computer system for performing the subject matter described in the instant disclosure. The illustrated computer (1002) is communicably coupled with a network (1030). In some implementations, one or more components of the computer (1002) may be configured to operate within environments, including cloud-computing-based, local, global, or other environment (or a combination of environments).

At a high level, the computer (1002) is an electronic computing device operable to receive, transmit, process, store, or manage data and information associated with the described subject matter. According to some implementations, the computer (1002) may also include or be communicably coupled with an application server, e-mail server, web server, caching server, streaming data server, business intelligence (BI) server, or other server (or a combination of servers).

The computer (1002) can receive requests over network (1030) from a client application (for example, executing on another computer (1002)) and responding to the received requests by processing the said requests in an appropriate software application. In addition, requests may also be sent to the computer (1002) from internal users (for example, from a command console or by other appropriate access method), external or third-parties, other automated applications, as well as any other appropriate entities, individuals, systems, or computers.

Each of the components of the computer (1002) can communicate using a system bus (1003). In some implementations, any or all of the components of the computer (1002), both hardware or software (or a combination of hardware and software), may interface with each other or the interface (1004) (or a combination of both) over the system bus (1003) using an application programming interface (API) (1012) or a service layer (1013) (or a combination of the API (1012) and service layer (1013). The API (1012) may include specifications for routines, data structures, and object classes. The API (1012) may be either computer-language independent or dependent and refer to a complete interface, a single function, or even a set of APIs. The service layer (1013) provides software services to the computer (1002) or other components (whether or not illustrated) that are communicably coupled to the computer (1002). The functionality of the computer (1002) may be accessible for all service consumers using this service layer. Software services, such as those provided by the service layer (1013), provide reusable, defined business functionalities through a defined interface. For example, the interface may be software written in JAVA, C++, or other suitable language providing data in extensible markup language (XML) format or another suitable format. While illustrated as an integrated component of the computer (1002), alternative implementations may illustrate the API (1012) or the service layer (1013) as stand-alone components in relation to other components of the computer (1002) or other components (whether or not illustrated) that are communicably coupled to the computer (1002). Moreover, any or all parts of the API (1012) or the service layer (1013) may be implemented as child or sub-modules of another software module, enterprise application, or hardware module without departing from the scope of this disclosure.

The computer (1002) includes an interface (1004). Although illustrated as a single interface (1004) in FIG. 10, two or more interfaces (1004) may be used according to particular needs, desires, or particular implementations of the computer (1002). The interface (1004) is used by the computer (1002) for communicating with other systems in a distributed environment that are connected to the network (1030). Generally, the interface (1004) includes logic encoded in software or hardware (or a combination of software and hardware) and operable to communicate with the network (1030). More specifically, the interface (1004) may include software supporting one or more communication protocols associated with communications such that the network (1030) or interface's hardware is operable to communicate physical signals within and outside of the illustrated computer (1002).

The computer (1002) includes at least one computer processor (1005). Although illustrated as a single computer processor (1005) in FIG. 10, two or more processors may be used according to particular needs, desires, or particular implementations of the computer (1002). Generally, the computer processor (1005) executes instructions and manipulates data to perform the operations of the computer (1002) and any algorithms, methods, functions, processes, flows, and procedures as described in the instant disclosure.

The computer (1002) also includes a memory (1006) that holds data for the computer (1002) or other components, such as computer executable instructions, (or a combination of both) that can be connected to the network (1030). The memory (1006) may be non-transitory computer readable memory. For example, memory (1006) can be a database storing data consistent with this disclosure. Although illustrated as a single memory (1006) in FIG. 10, two or more memories may be used according to particular needs, desires, or particular implementations of the computer (1002) and the described functionality. While memory (1006) is illustrated as an integral component of the computer (1002), in alternative implementations, memory (1006) can be external to the computer (1002).

The application (1007) is an algorithmic software engine providing functionality according to particular needs, desires, or particular implementations of the computer (1002), particularly with respect to functionality described in this disclosure. For example, application (1007) can serve as one or more components, modules, applications, etc. Further, although illustrated as a single application (1007), the application (1007) may be implemented as multiple applications (1007) on the computer (1002). In addition, although illustrated as integral to the computer (1002), in alternative implementations, the application (1007) can be external to the computer (1002).

There may be any number of computers (1002) associated with, or external to, a computer system containing computer (1002), wherein each computer (1002) communicates over network (1030). Further, the term “client,” “user,” and other appropriate terminology may be used interchangeably as appropriate without departing from the scope of this disclosure. Moreover, this disclosure contemplates that many users may use one computer (1002), or that one user may use multiple computers (1002).

Although only a few example embodiments have been described in detail above, those skilled in the art will readily appreciate that many modifications are possible in the example embodiments without materially departing from this invention. In addition, many modifications will be appreciated by those skilled in the art to adapt a particular instrument, situation, or material to embodiments of the disclosure without departing from the essential scope thereof. Accordingly, all such modifications are intended to be included within the scope of this disclosure as defined in the following claims. Moreover, reference in the appended claims to an apparatus or system or a component of an apparatus or system being adapted to, arranged to, capable of, configured to, enabled to, operable to, or operative to perform a particular function encompasses that apparatus, system, or component, whether or not it or that particular function is activated, turned on, or unlocked, as long as that apparatus, system, or component is so adapted, arranged, capable, configured, enabled, operable, or operative.

Furthermore, any apparatus described herein may be free of any component not expressly recited or disclosed herein. Any method or system may lack any step not recited or disclosed herein. Likewise, the term “comprising” is considered synonymous with the term “including.” Whenever a method, composition, element or group of elements is preceded with the transitional phrase “comprising,” it is understood that we also contemplate the same composition or group of elements with transitional phrases “consisting essentially of,” “consisting of,” “selected from the group of consisting of,” or “is” preceding the recitation of the composition, element, or elements and vice versa.

Unless otherwise indicated, all numbers expressing quantities of ingredients, properties such as molecular weight, reaction conditions, and so forth used in the present specification and associated claims are to be understood as being modified in all instances by the term “about.” Accordingly, unless indicated to the contrary, the numerical parameters set forth in the following specification and attached claims are approximations that may vary depending upon the desired properties sought to be obtained by one or more embodiments described herein. At the very least, and not as an attempt to limit the application of the doctrine of equivalents to the scope of the claim, each numerical parameter should at least be construed in light of the number of reported significant digits and by applying ordinary rounding techniques.

What is claimed is:

1. A system, comprising:
a molecular descriptor generator configured to receive a simplified molecular-input line-entry system (SMILES) representation of a molecule and output a molecular descriptor for the molecule;
a pre-processor configured to receive the molecular descriptor from the molecular descriptor generator and output a pre-processed molecular descriptor, wherein the pre-processor comprises a set of previously determined pre-processor parameters;
a trained chemical super learner model configured to receive the pre-processed molecular descriptor from the pre-processor and output a property prediction for the molecule, wherein the trained chemical super learner model is composed of a weighted average of one or more trained machine-learned models; and
a computer, the computer comprising:
one or more computer processors configured to:
receive a first production example comprising a SMILES representation of a first molecule;
process the first production example with the molecular descriptor generator to produce a first molecular descriptor;
pre-process, with the pre-processor, the first molecular descriptor;
process, with the trained chemical super learner model, the pre-processed first molecular descriptor to determine a first prediction for a property of the first molecule.

2. The system of claim 1, wherein the one or more processors are further configured to;
receive a second production example comprising a SMILES representation of a second molecule;
process the second production example with the molecular descriptor generator to produce a second molecular descriptor;
pre-process, with the pre-processor, the second molecular descriptor;
process, with the trained chemical super learner model, the pre-processed second molecular descriptor to determine a second prediction for the property of the second molecule.

3. The system of claim 1, further comprising an inversion system configured to, at least, determine a prediction of the property of a molecule by sequentially using the molecular descriptor, pre-processor, and trained chemical super learner model.

4. The system of claim 3, wherein the one or more computer processors are further configured to:
obtain a plurality of production examples from a production database wherein each production example comprises a SMILES representation of a unique molecule;
process the plurality of production examples with the molecular descriptor generator to produce a plurality of molecular descriptors;
pre-process, with the pre-processor, the plurality of molecular descriptors; and
iteratively, until stopped by a stopping criterion:
select, with the inversion system, a production example from the plurality production examples, and
process, with the trained chemical super learner model, the pre-processed molecular descriptor of the selected production example to determine a selected property prediction;
wherein the inversion system selects the production example based on the property prediction of all production examples previously processed by the trained chemical super learner model.

5. A computer-implemented method of training a chemical super learner model, comprising:
obtaining a plurality of training examples from a training database wherein each training example comprises:
a simplified molecular-input line-entry system (SMILES) description of a molecule, and
a first property;
processing the plurality of training examples, with a molecular descriptor generator to produce a plurality of molecular descriptors;
pre-processing, with a pre-processor, the plurality of molecular descriptors;
training one or more machine-learned models using the pre-processed plurality of molecular descriptors and the training database, wherein each of the one or more machine-learned models are configured to accept a pre-processed molecular descriptor and return a first property prediction;
scoring the one or more machine learned models, wherein upon scoring each of the one or more machine-learned models has a score;
selecting a subset of the one or more machine learned models, wherein each of the machine-learned models in the subset has a better score than the machine-learned models outside of the subset;
tuning hyperparameters of each of the machine-learned models in the subset;
determining a weight for each machine-learned model in the subset; and
forming the trained chemical super learner model as a weighted average of each machine-learned model in the subset, wherein each machine-learned model in the subset is weighted in the weighted average according to its weight.

6. The method of claim 5 wherein each of the training examples in the plurality of training examples further comprises a second property.

7. The method of claim 6 wherein each of the one or more machine-learned models are trained jointly with the first property and the second property and configured to return a first property prediction and a second property prediction.

8. The method of claim 5, wherein the pre-processor comprises a set of pre-processor parameters.

9. The method of claim 5, wherein the molecular descriptor generator accepts the SMILES of each training example in the plurality of training examples and returns a vector for each training example, the vector comprising:
a Morgan fingerprint representation of the training example;
a Mordred representation of the training example; and
an embedding representation of the training example.

10. The method of claim 5, wherein the hyperparameters of each machine-learned model in the subset are tuned independently using a genetic algorithm.

11. The method of claim 5, wherein the weight of each of the machine-learned models in the subset is determined using a sequential least-squares programming meta learner.

12. The method of claim 5, further comprising estimating a generalization error of the trained chemical super learner model.

13. A non-transitory computer readable medium storing instructions executable by a computer processor, the instructions comprising functionality for:
obtaining a plurality of training examples from a training database wherein each training example comprises:
a simplified molecular-input line-entry system (SMILES) description of a molecule, and
a first property;
processing the plurality of training examples, with a molecular descriptor generator to produce a plurality of molecular descriptors;
pre-processing, with a pre-processor, the plurality of molecular descriptors;
training one or more machine-learned models using the pre-processed plurality of molecular descriptors and the training database, wherein each of the one or more machine-learned models are configured to accept a pre-processed molecular descriptor and return a first property prediction;
scoring the one or more machine learned models, wherein upon scoring each of the one or more machine-learned models has a score;
selecting a subset of the one or more machine learned models, wherein each of the machine-learned models in the subset has a better score than the machine-learned models outside of the subset;
tuning hyperparameters of each of the machine-learned models in the subset;
determining a weight for each machine-learned model in the subset; and
forming a trained chemical super learner model as a weighted average of each machine-learned model in the subset, wherein each machine-learned model in the subset is weighted in the weighted average according to its weight.
obtaining a first production example from a production database;
processing the first production example with the molecular descriptor generator to produce a first molecular descriptor;
pre-processing, with the pre-processor, the first molecular descriptor;
processing the trained chemical super learner model to predict the first property for the first production example.

14. The non-transitory computer readable medium of claim 13 wherein each of the training examples in the plurality of training examples further comprises a second property.

15. The non-transitory computer readable medium of claim 14 wherein each of the one or more machine-learned models are trained jointly with the first property and the second property and configured to return a first property prediction and a second property prediction.

16. The non-transitory computer readable medium of claim 13, wherein the pre-processor comprises a set of pre-processor parameters.

17. The non-transitory computer readable medium of claim 13, wherein the molecular descriptor generator accepts the SMILES of each training example in the plurality of training examples and returns a vector for each training example, the vector comprising:
a Morgan fingerprint representation of the training example;
a Mordred representation of the training example; and
an embedding representation of the training example.

18. The non-transitory computer readable medium of claim 13, wherein the hyperparameters of each machine-learned model in the subset are tuned independently using a genetic algorithm.

19. The non-transitory computer readable medium of claim 13, wherein the weight of each of the machine-learned models in the subset is determined using a sequential least-squares programming meta learner.

20. The non-transitory computer readable medium of claim 13, the instructions further comprising estimating a generalization error of the trained chemical super learner model.
